(19) United States
(12) Patent Application Publication
(10) Pub. No.: US 2008/0002834 A1
(43) Pub. Date: Jan. 3, 2008

(54) Button Processor For Cochlear Implants

(76) Inventor: Erwin Hochmair, Axams (AT)

Correspondence Address: BROMBERG & SUNSTEIN LLP, 125 SUMMER STREET, BOSTON, MA, 02110-1618, US

(21) Appl. No.: 11/760,131

(22) Filed: Jun. 8, 2007

Related U.S. Application Data
(60) Provisional application No. 60/812,431, filed on Jun. 9, 2006.

(51) Int. Cl. A61F 11/06

(52) U.S. Cl. 381/71.2

(57) ABSTRACT

A cochlear implant system has an external housing adapted for placement at a specific location on the external skin of a user. The external housing includes (i) a signal processing module for converting an incoming acoustic signal into a representative electrical information signal, and (ii) a signal transmission module for transmitting across the skin of the user an electrical power signal representative of the electrical information signal. An implanted portion receives the electrical power signal and produces for the auditory system of the user an electrical stimulation signal representative of the acoustic signal. The external housing and the implanted portion magnetically cooperate so as to hold the external housing in place at the specific location without structural interaction with the outer ear of the user.

### FIELD OF THE INVENTION

The invention generally relates to cochlear implant systems, and specifically to the external structures of such systems.

### BACKGROUND ART

A normal ear transmits sounds as shown in FIG. 1 through the outer ear 101 to the eardrum 102, which moves the bones of the middle ear 103, which in turn excites the cochlea 104. The cochlea 104 includes an upper channel known as the scala vestibuli 105 and a lower channel known as the scala tympani 106, which are connected by the cochlear duct 107. In response to received sounds transmitted by the middle ear 103, the fluid filled scala vestibuli 105 and scala tympani 106 function as a transducer to transmit waves to generate electric pulses that are transmitted to the cochlear nerve 113, and ultimately to the brain.

Some persons have partial or full loss of normal sensorineural hearing. Cochlear implant systems have been developed to overcome this by directly stimulating the user's cochlea 104. A typical system may include an external microphone that provides an audio signal input to an external signal processing stage (not shown in FIG. 1) where various signal processing schemes can be implemented. The processed signal is then converted into a digital data format, such as a sequence of data frames, for transmission into receiver 108. Besides extracting the audio information, the receiver 108 also performs additional signal processing such as error correction, pulse formation, etc., and produces a stimulation pattern (based on the extracted audio information) that is sent through connected wires 109 to an implanted electrode carrier 110. Typically, this electrode carrier 110 includes multiple electrodes on its surface that provide selective stimulation of the cochlea 104.

Existing cochlear implant systems need to deliver electrical power from outside the body through the skin to satisfy the power requirements of the implanted portion of the system. FIG. 1 shows a typical arrangement based on inductive coupling through the skin to transfer both the required electrical power and the processed audio information. As shown in FIG. 1, an external primary coil 111 (coupled to the external signal processor) is placed on the skin adjacent to a subcutaneous secondary coil 112 (connected to the receiver 108). Often, a magnet in the external coil structure interacts a corresponding magnet in the subcutaneous secondary coil structure. This arrangement inductively couples a radio frequency (rf) electrical signal to the receiver 108. The receiver 108 is able to extract from the rf signal both the audio information for the implanted portion of the system and a power component to power the implanted system.

In prior systems, the external components generally have been held in separate housings so that the external primary coil 111 would not be in the same physical housing as the power source or the external signal processor. The various different physical components would generally be connected by hard wire, although some systems used wireless links between separate external components. A few systems have proposed that all of the external components could be placed within a single housing, but the weight of the various required components was relatively significant and such systems had to rely on anchoring such a single external housing on the stiff cartilaginous outer ear in order to provide enough support to hold the housing securely in a usable operating position.

### SUMMARY OF THE INVENTION

A representative embodiment of the present invention includes a cochlear implant system having an external housing adapted for placement at a specific location on the external skin of a user. The external housing includes (i) a signal processing module for converting an incoming acoustic signal into a representative electrical information signal, and (ii) a signal transmission module for transmitting across the skin of the user an electrical power signal representative of the electrical information signal. An implanted portion receives the electrical power signal and produces for the auditory system of the user an electrical stimulation signal representative of the acoustic signal. The external housing and the implanted portion magnetically cooperate so as to hold the external housing in place at the specific location without structural interaction with the outer ear of the user.

In specific embodiments, the signal transmission module may include a transcutaneous transdermal transmission coil for transcutaneously transmitting the electrical power signal. Similarly, the implanted portion may include a reception coil for receiving the electrical power signal. The electrical power signal may be less than 20 mW and/or the external housing may weigh less than 10 g. The external housing may further include at least one battery, such as a Li-polymer battery or a zinc air battery.

Embodiments also include a method of operating a cochlear implant system. An external housing adapted for placement at a specific location on the external skin of a user is provided. The external housing includes (i) a signal processing module for converting an incoming acoustic signal into a representative electrical information signal, and (ii) a signal transmission module for transmitting across the skin of the user an electrical power signal representative of the electrical information signal. The electrical power signal is received with an implanted portion. And an electrical stimulation signal representative of the acoustic signal is produced for the auditory system of the user. The external housing and the implanted portion magnetically cooperate so as to hold the external housing in place at the specific location without structural interaction with the outer ear of the user.

In further specific embodiments, transmitting the electrical power signal may use a transmitting coil arrangement within the external housing. Similarly, receiving the electrical power signal may use a receiving coil arrangement associated with the implanted portion. The electrical power signal may be less than 20 mW and/or the external housing may weigh less than 10 g. The source of the electrical power signal may be at least one battery within the external housing such as a Li-polymer battery or a zinc air battery.

### BRIEF DESCRIPTION OF THE DRAWINGS

FIG. 1 shows the ear structure of a human ear and a typical cochlear implant system.

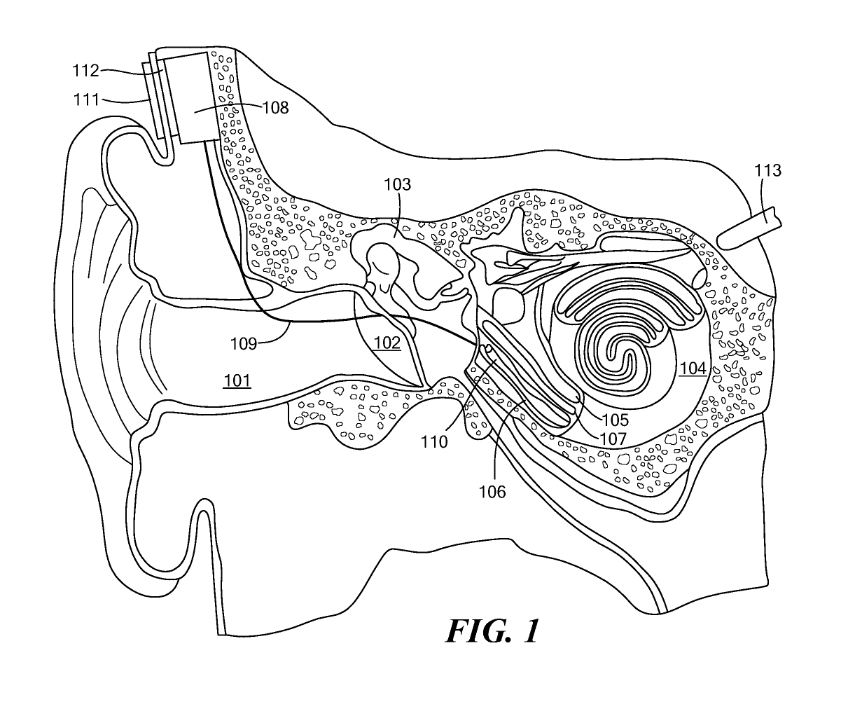

FIG. 2 shows the outlines of a single external housing in place behind the ear of a user according to an embodiment of the present invention.

FIG. 3 shows a cross-sectional function view of one specific embodiment of the present invention.

### DETAILED DESCRIPTION OF SPECIFIC EMBODIMENTS

Embodiments of the present invention are directed to a cochlear implant system having an external component in a single housing. The external housing is held in place at a specific location for proper electrical interaction with the implanted components. Unlike prior art systems which combined all external components into a single housing, embodiments of the present invention are designed to be light enough to be held in place without structural interaction with the outer ear of the user, solely by use of transdermal magnetics.

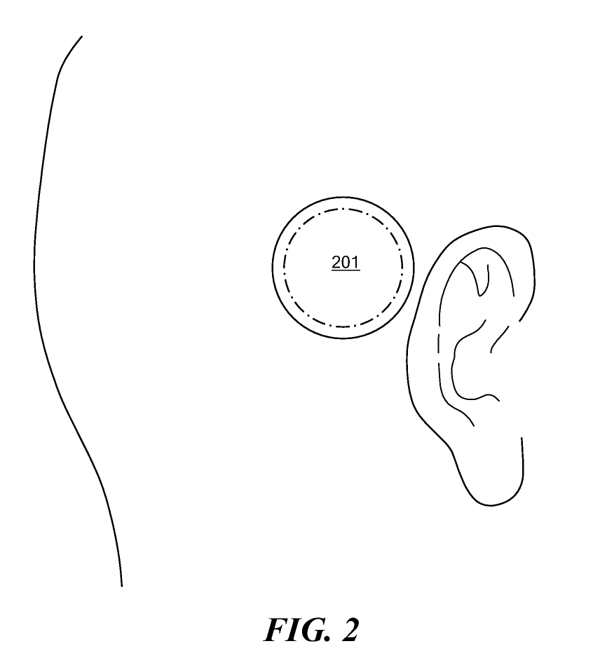

FIG. 2 shows the outlines of such a single external housing 201 held in place on the skin of the user behind the ear without relying on a mechanical connection to the ear to provide support and anchoring for the housing. By controlling the power consumption requirements of the implanted portion of the system, efficiently generating the transdermal power and information signal into the implanted portion, utilizing light weight efficient batteries and structural materials, the entire external housing is overall light enough that magnetic coupling alone is capable of supporting the structure and maintaining it in place. For example, in specific embodiments the external housing may weigh 10 grams or less.

In contrast with ear-supported single housing systems, embodiments of the present invention are more comfortable for the user because they do not tug on the outer ear. Embodiments may also be more easily concealed beneath the hair of the wearer so that user may feel less self-conscious about using a cochlear implant.

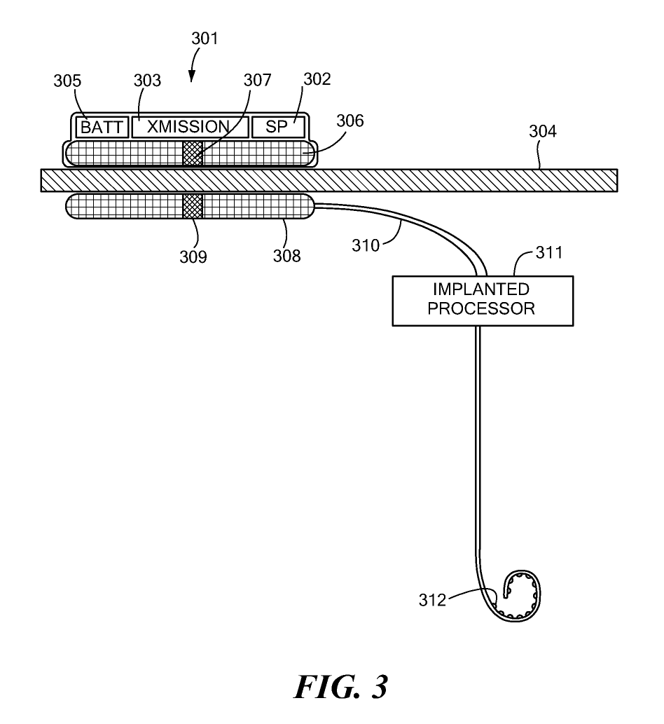

FIG. 3 shows a cross-sectional function view of one specific embodiment of the present invention. An external housing 301 includes a signal processing module 302 for converting an incoming acoustic signal into a representative electrical information signal. The signal processing module 302 may include a microphone and related input signal processing circuitry and also various signal conditioning and power conditioning signal circuitry. The external housing 301 also includes a signal transmission module 303 powered by one or more batteries 305 for transmitting across the skin 304 of the user an electrical power signal representative of the electrical information signal. The one or more batteries 305 may typically be any modern lightweight efficient battery such as a Li-polymer battery or a zinc air battery.

The portion of the external housing 301 next to the skin 304 includes a primary transmission coil 306 and external positioning magnet 307. Directly beneath these and under the skin 304 are a corresponding secondary receiving coil 308 and internal positioning magnet 309. The magnetic coupling between the external positioning magnet 307 and the internal positioning magnet 309 is strong enough to hold in the external housing 301 in proper position on the skin 304 of the user directly over the secondary receiving coil 308. An electrical power signal including the electrical information representative of the input acoustic signal is developed by the primary transmission coil 306 and coupled across the skin 304 to the secondary receiving coil 308.

The electrical power signal received by the secondary receiving coil 308 is, e.g., coupled by implanted wires 310 to an implanted signal processor 311 which produces an electrical stimulation signal representative of the acoustic signal for an implanted stimulation electrode 312 to stimulate the auditory nerves of the cochlea. In another embodiment, the receiving coil 308 may be integrated within the case of the implanted signal processor 311.

By efficient internal processing and stimulation, the power requirements of the implanted portion may be minimized. This allows a relatively low power electrical power signal to be used, for example, the electrical power signal may be less than 20 milliwatts. Such low power requirements contribute to allowing the external housing 301 to be as light and efficient as possible, enough so as to enable the external housing 301 to be maintained in proper position solely by the positioning magnetics without relying on physical support from the outer ear.

Although various exemplary embodiments of the invention have been disclosed, it should be apparent to those skilled in the art that various changes and modifications can be made which will achieve some of the advantages of the invention without departing from the true scope of the invention.

What is claimed is:

1. A cochlear implant system comprising:
an external housing adapted for placement at a specific location on the external skin of a user, the external housing including:
i. a signal processing module for converting an incoming acoustic signal into a representative electrical information signal, and
ii. a signal transmission module for transmitting across the skin of the user an electrical power signal representative of the electrical information signal; and
an implanted portion that receives the electrical power signal and produces for the auditory system of the user an electrical stimulation signal representative of the acoustic signal,
wherein the external housing and the implanted portion magnetically cooperate so as to hold the external housing in place at the specific location without structural interaction with the outer ear of the user.

2. A system according to claim 1, wherein the signal transmission module includes a transdermal transmission coil for transmitting the electrical power signal.

3. A system according to claim 1, wherein the implanted portion includes a reception coil for receiving the electrical power signal.

4. A system according to claim 1, wherein the electrical power signal is less than 20 mW.

5. A system according to claim 1, wherein the external housing weighs less than 10 g.

6. A system according to claim 1, wherein the external housing further includes at least one battery.

7. A system according to claim 6, wherein the at least one battery is a Li-polymer battery.

8. A system according to claim 6, wherein the at least one battery is a zinc air battery.

9. A method of operating a cochlear implant system, the method comprising:
providing an external housing adapted for placement at a specific location on the external skin of a user, the external housing including:
i. a signal processing module for converting an incoming acoustic signal into a representative electrical information signal, and
ii. a signal transmission module for transmitting across the skin of the user an electrical power signal representative of the electrical information signal; and receiving the electrical power signal with an implanted portion, and
producing for the auditory system of the user an electrical stimulation signal representative of the acoustic signal,
wherein the external housing and the implanted portion magnetically cooperate so as to hold the external housing in place at the specific location without structural interaction with the outer ear of the user.

10. A method according to claim 9, wherein transmitting the electrical power signal uses a transmitting coil arrangement within the external housing.

11. A method according to claim 9, wherein receiving the electrical power signal uses a receiving coil arrangement associated with the implanted portion.

12. A method according to claim 9, wherein the electrical power signal is less than 20 mW.

13. A method according to claim 9, wherein the external housing weighs less than 10 g.

14. A method according to claim 9, wherein the source of the electrical power signal is at least one battery within the external housing.

15. A method according to claim 14, wherein the at least one battery is a Li-polymer battery.

16. A method according to claim 14, wherein the at least one battery is a zinc air battery.
